(19) United States
(12) Patent Application Publication
(10) Pub. No.: US 2007/0066940 A1
(43) Pub. Date: Mar. 22, 2007

(54) Systems and Methods for Detecting a Partition Position in an Infusion Pump

(75) Inventors: Arjuna Karunaratne, Fremont, CA (US); Anthony Lam, Fremont, CA (US); Mingqi Zhao, San Jose, CA (US); Braddon M. Van Slyke, Arvada, CO (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: LifeScan, Inc., Milpitas, CA (US)

(21) Appl. No.: 11/532,653

(22) Filed: Sep. 18, 2006

Related U.S. Application Data
(60) Provisional application No. 60/718,572, filed on Sep. 19, 2005.
(60) Provisional application No. 60/718,397, filed on Sep. 19, 2005.
(60) Provisional application No. 60/718,412, filed on Sep. 19, 2005.
(60) Provisional application No. 60/718,577, filed on Sep. 19, 2005.
(60) Provisional application No. 60/718,578, filed on Sep. 19, 2005.
(60) Provisional application No. 60/718,364, filed on Sep. 19, 2005.
(60) Provisional application No. 60/718,399, filed on Sep. 19, 2005.
(60) Provisional application No. 60/718,400, filed on Sep. 19, 2005.
(60) Provisional application No. 60/718,398, filed on Sep. 19, 2005.
(60) Provisional application No. 60/718,289, filed on Sep. 19, 2005.

(51) Int. Cl. A61M 1/00

(52) U.S. Cl. 604/152

(57) ABSTRACT

An infusion pump (e.g., an electrokinetic infusion pump) includes an infusion pump module and an engine that can drive a moveable piston non-mechanically. In addition, the infusion pump module includes a position detector configured for sensing a dispensing state of the infusion pump module. Such information can be utilized in a control scheme to control fluid displacement within and out of the pump. Descriptions of different types of position detectors, such as magnetic sensors (e.g., an anisotropic magnetic resistive sensor), and their implementation in detecting infusion pump fluid displacement are described.

### CROSS REFERENCE TO RELATED APPLICATIONS

The present application claims the benefit of the following U.S. Provisional Applications, all filed on Sep. 19, 2005: Ser. No. 60/718,572, bearing attorney docket number LFS-5093USPSP and entitled “Electrokinetic Infusion Pump with Detachable Controller and Method of Use”; Ser. No. 60/718,397, bearing attorney docket number LFS-5094USPSP and entitled “A Method of Detecting Occlusions in an Electrokinetic Pump Using a Position Sensor”; Ser. No. 60/718,412, bearing attorney docket number LFS-5095USPSP and entitled “A Magnetic Sensor Capable of Measuring a Position at an Increased Resolution”; Ser. No. 60/718,577, bearing attorney docket number LFS-5096USPSP and entitled “A Drug Delivery Device Using a Magnetic Position Sensor for Controlling a Dispense Rate or Volume”; Ser. No. 60/718,578, bearing attorney docket number LFS-5097USPSP and entitled “Syringe-Type Electrokinetic Infusion Pump and Method of Use”; Ser. No. 60/718,364, bearing attorney docket number LFS-5098USPSP and entitled “Syringe-Type Electrokinetic Infusion Pump for Delivery of Therapeutic Agents”; Ser. No. 60/718,399, bearing attorney docket number LFS-5099USPSP and entitled “Electrokinetic Syringe Pump with Manual Prime Capability and Method of Use”; Ser. No. 60/718,400, bearing attorney docket number LFS-5100USPSP and entitled “Electrokinetic Pump Integrated within a Plunger of a Syringe Assembly”; Ser. No. 60/718,398, bearing attorney docket number LFS-5101USPSP and entitled “Reduced Size Electrokinetic Pump Using an Indirect Pumping Mechanism with Hydraulic Assembly”; and Ser. No. 60/718,289, bearing attorney docket number LFS-5102USPSP and entitled “Manual Prime Capability of an Electrokinetic Syringe Pump and Method of Use.” The present application is also related to the following applications, all filed concurrently herewith: “Electrokinetic Infusion Pump System” (Attorney Docket No.106731-5); “Infusion Pump with Closed Loop Control and Algorithm” (Attorney Docket No. 106731-3); “Malfunction Detection via Pressure Pulsation” (Attorney Docket No. 106731-6); “Infusion Pumps with a Position Detector” (Attorney Docket No. 106731-18); and “Malfunction Detection with Derivative Calculation” (Attorney Docket No. 106731-22). All of the applications recited in this paragraph are hereby incorporated by reference herein in their entirety.

### FIELD OF THE INVENTION

The present invention relates, in general, to medical devices and systems and, in particular, to infusion pumps, infusion pump systems and associated methods.

### BACKGROUND OF THE INVENTION

Electrokinetic pumps provide for liquid displacement by applying an electric potential across a porous dielectric media that is filled with an ion-containing electrokinetic solution. Properties of the porous dielectric media and ion-containing solution (e.g., permittivity of the ion-containing solution and zeta potential of the solid-liquid interface between the porous dielectric media and the ion-containing solution) are predetermined such that an electrical double-layer is formed at the solid-liquid interface. Thereafter, ions of the electrokinetic solution within the electrical double-layer migrate in response to the electric potential, transporting the bulk electrokinetic solution with them via viscous interaction. The resulting electrokinetic flow (also known as electroosmotic flow) of the bulk electrokinetic solution is employed to displace (i.e., “pump”) a liquid. Further details regarding electrokinetic pumps, including materials, designs, and methods of manufacturing are included in U.S. patent application Ser. No. 10/322,083 filed on Dec. 17, 2002, which is hereby incorporated in full by reference.

### SUMMARY OF THE INVENTION

One exemplary embodiment is directed to a method of locating a moveable partition's location for an infusion pump using one or more displacement sensors, such as sensors that can provide a signal based at least in part upon the partition's position (e.g., sensors that can detect a magnetic field such as an anisotropic magnetic resistive sensor). A potential range of moveable partition positions can be selected, and the range can be segmented into a set of potential positions (e.g., a set of equally spaced potential positions). Selection of the potential range can be based upon using a last designated position of the moveable partition, and can further include selecting a distance before and after the last designated position. A set of error measures can be calculated, with each error measure corresponding to one potential position in the potential range. Each error measure can be based at least in part upon an actual displacement sensor signal from at least one displacement sensor and the potential position to which the error measure is associated. A new partition position can be chosen from the set of potential positions by setting the new position equal to the potential position associated with the lowest calculated error measure in the set of error measures. The new moveable partition position can be used to determine an amount of fluid displacement within or from the infusion pump based upon the displacement of the partition (e.g., the difference between the new position and a former position). The new partition position can also be used in a closed loop control algorithm to control subsequent fluid delivery. The exemplary embodiment can be used on a variety of infusion pumps such as infusion pumps with an electrokinetic engine, and/or generally those utilizing a non-mechanically-driven moveable partition.

An error measure, for the exemplary embodiment, can be determined, at least in part, by calculating a measure of a difference between the actual displacement sensor signal and a predicted displacement sensor signal for at least one potential position in the potential range. Predicted displacement sensor signals for each sensor can be provided by a calibrated model, such as a fitted polynomial. In one instance, each error measure at a potential position can be a mean square error, which can be found by summing the squares of a set of calculated differences between the actual displacement sensor signal and a predicted displacement sensor signal for each of the displacement sensors, the predicted displacement sensor signal depending at least in part on the potential position. In other instances, not all of the sensor signals are utilized in calculating an error measure when a plurality of sensors are used in an infusion pump. For example, only the two displacement sensors located closest to the moveable partition (e.g., the last known position of the partition could be used) can be employed.

In a potential aspect of the exemplary embodiment, a lowest error measure in a set of error measures associated with a potential range of partition positions can be identified according to the following steps. An error measure is calculated at a current potential position for the moveable partition. A candidate position of the moveable partition can be set equal to either the current potential position or a previously calculated potential position depending upon the error measures associated with the positions (e.g., choosing the potential position with the lower error measure). These steps can be repeated for each of the potential positions in the range, and the new partition position can be set equal to the last candidate position value.

In accord with the exemplary embodiment, the steps of the method can be repeated as the moveable partition proceeds through the infusion pump. In particular, after each successive repetition of the steps, new actual sensor signals can be obtained for use with the subsequent repetition of the steps. Alternatively, the steps can be repeated using a particular set of actual sensor signals. Each successive repetition of steps can segment a corresponding potential range of positions into equally spaced potential positions that are closer together, with the corresponding potential range becoming smaller with each successive repetition of steps. For example, each successive repetition of steps can reduce the corresponding potential range by a factor of at least about two, and/or reduce the segmentation spacing between potential positions by a factor of at least about two.

Another exemplary embodiment is directed toward a system for locating a position of a moveable partition in an infusion pump that includes a magnet coupled to the moveable partition, and one or more magnetic sensors (e.g., anisotropic magnetic resistive sensors). The magnetic sensors can be coupled to the infusion pump's body (e.g., at least two magnetic sensors disposed along a distance traversable by the partition). Each of the magnetic sensors can emit a signal when subjected to a magnetic field. The system can also include a processor coupled to each of the magnetic sensors.

The processor of the system can be configured to carry out any of the functionalities described by embodiments described herein. For example, the processor can be configured to identify the position of the moveable partition at least in part by calculating a set of error measures over a potential range of positions. The set of error measures can depend in part upon at least one actual sensor measurement and a set of potential positions within the potential range. The processor can be configured to identify a moveable partition's position by equating it with a corresponding potential position having a lowest error measurement. Furthermore, the processor can be configured to calculate the set of error measures based upon any of the techniques described herein.

The system can further include a memory configured to store data utilized to identify a predicted sensor signal for a magnetic sensor at each of a set of potential positions that can be used to calculate error measures. For example, the memory can store the coefficients of a polynomial function that can model a sensor signal. The system can also include a closed loop controller that is coupled to the processor. Such a controller can receive a position from the processor and use the position to control fluid flow associated with the infusion pump.

### BRIEF DESCRIPTION OF THE DRAWINGS

FIG. 1 is a simplified, exploded schematic illustration of an electrokinetic infusion pump system with closed loop control according to an exemplary embodiment of the present invention in a first dispense state;

FIG. 2 is a simplified, exploded schematic illustration of the electrokinetic infusion pump system of FIG. 1 in a second dispense state;

FIG. 3 is a simplified perspective illustration of an electrokinetic infusion pump system according to another exemplary embodiment of the present invention being manually manipulated;

FIG. 4 is a simplified cross-sectional and schematic depiction of portions of an electrokinetic infusion pump according to a further exemplary embodiment of the present invention;

FIG. 5 is a simplified cross-sectional depiction of an electrokinetic infusion pump system according to an additional exemplary embodiment of the present invention in a first dispense state;

FIG. 6 is a simplified cross-sectional depiction of the electrokinetic infusion pump system of FIG. 5 in a second dispense state;

FIG. 7 is a graph of shot size versus time obtained using an experimental electrokinetic infusion pump system in accord with an embodiment of the present invention;

FIG. 8 is a graph of linear range and resolution versus gap for other experimental electrokinetic infusion pumps in accord with an embodiment of the present invention;

FIG. 9 is a flow diagram illustrating a method for the closed loop control of an electrokinetic infusion pump according to an exemplary embodiment of the present invention;

FIG. 10 is an illustration of a magnetic linear position detector as can be used with an electrokinetic infusion pump according to an embodiment of the present invention;

FIGS. 11A and 11B illustrate portions of an electrokinetic infusion pump in two fluid dispensing states according to an embodiment of the present invention, including an electrokinetic engine, an infusion module, a magnetostrictive waveguide, and a position sensor control circuit;

FIG. 12A is a flow chart illustrating an algorithm for determining the position of a moveable partition of an infusion pump using one or more position sensor signals, in accord with an embodiment of the invention;

FIG. 12B is a flow chart illustrating an exemplary technique for calculating an error measure at a designated potential partition position in accord with the algorithm illustrated in FIG. 12A;

FIG. 12C is a flow chart illustrate an exemplary technique for identifying a potential position in a range of positions that is associated with a minimum error measure in accord with the algorithm illustrated in FIG. 12A; and

FIG. 13 is a schematic diagram of a system for locating a position of a moveable partition of an infusion pump, in accord with embodiments of the invention.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. It should also be understood that for the various steps of the methods discussed herein, the order of the steps need not follow the description's order of describing the steps, unless otherwise explicitly stated. Such modifications and variations are intended to be included within the scope of the present invention.

Electrokinetic Infusion Pump Systems

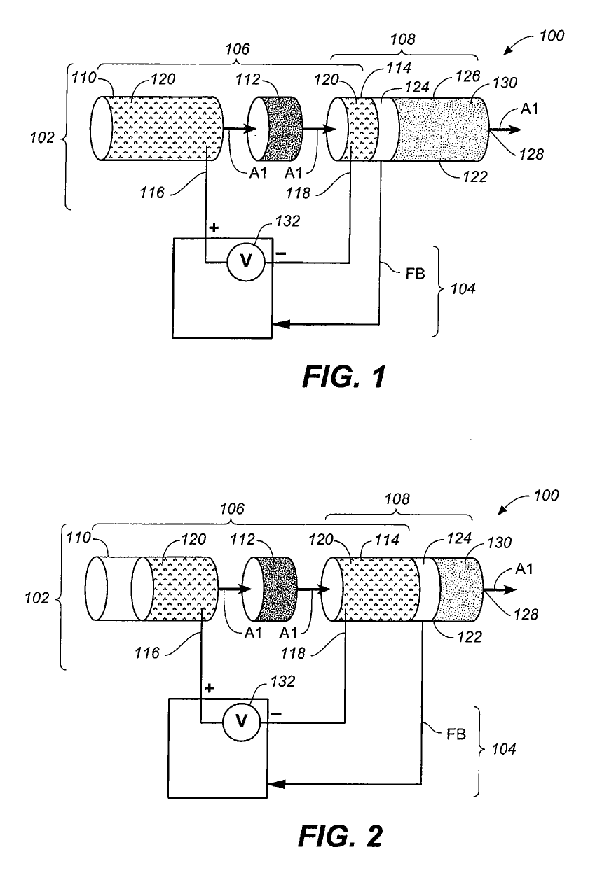

FIG. 1 is a simplified, exploded schematic illustration of an electrokinetic infusion pump system 100 with closed loop control according to an exemplary embodiment of the present invention in a first dispense state, while FIG. 2 depicts electrokinetic infusion pump system 100 in a second dispense state.

Referring to FIGS. 1 and 2, the depicted electrokinetic infusion pump system 100 includes an electrokinetic infusion pump 102 and a closed loop controller 104. Electrokinetic infusion pump 102 includes a position detector (not shown in FIGS. 1 and 2). As is described in further detail below, electrokinetic infusion pump 102 and closed loop controller 104 are in operative communication such that closed loop controller 104 can determine and control the dispensing state of electrokinetic infusion pump 102 based on a feedback signal(s) FB from the position detector. Electrokinetic infusion pump 102 and closed loop controller 104 can be entirely separate units, partially integrated (for example, predetermined components of electrokinetic infusion pump 102 can be integrated within closed loop controller 104) or a single integrated unit.

Electrokinetic infusion pump systems according to embodiments of the present invention, including electrokinetic infusion pump system 100, can be employed to deliver a variety of medically useful infusion liquids such as, for example, insulin for diabetes; morphine and other analgesics for pain; barbiturates and ketamine for anesthesia; anti-infective and antiviral therapies for Acquired Immune Deficiency Syndrome (AIDS); antibiotic therapies for preventing infection; bone marrow for immunodeficiency disorders, blood-borne malignancies, and solid tumors; chemotherapy for cancer; dobutamine for congestive heart failure; monoclonal antibodies and vaccines for cancer, brain natiuretic peptide for congestive heart failure, and vascular endothelial growth factor for preeclampsia. The delivery of such infusion liquids can be accomplished via any suitable route including subcutaneously, intravenously or intraspinally.

Electrokinetic infusion pump 102 includes an electrokinetic engine 106 and an infusion module 108. Electrokinetic engine 106 includes an electrokinetic supply reservoir 110, electrokinetic porous media 112, electrokinetic solution receiving chamber 114, first electrode 116, second electrode 118 and electrokinetic solution 120 (depicted as upwardly pointing chevrons).

The pore size of porous media 112 can be, for example, in the range of 100 nm to 200 nm. Moreover, porous media 112 can be formed of any suitable material including, for example, Durapore Z PVDF membrane material available from Millipore, Inc. USA. Electrokinetic solution 120 can be any suitable electrokinetic solution including, but not limited to, 10 mM TRIS/HCl at a neutral pH.

Infusion module 108 includes electrokinetic solution receiving chamber 114 (which is also considered part of electrokinetic engine 106), infusion module housing 122, movable partition 124, infusion reservoir 126, infusion reservoir outlet 128 and infusion liquid 130 (depicted as dotted shading). Although the position detector of infusion module 108 is not depicted in FIGS. 1 and 2, feedback signal FB between the position detector and closed loop controller 104 is shown.

Closed loop controller 104 includes voltage source 132 and is configured to receive feedback signal FB from the position detector and to be in electrical communication with first and second electrodes 116 and 118. Electrokinetic engine 106, infusion module 108 and closed loop controller 104 can be integrated into a single assembly, into multiple assemblies or can be separate units.

During operation of electrokinetic infusion pump system 100, electrokinetic engine 106 provides the driving force for displacing (pumping) infusion liquid 130 from infusion module 108. To do so, a voltage difference is established across electrokinetic porous media 112 by the application of an electrical potential between first electrode 116 and second electrode 118. This electrical potential results in an electrokinetic pumping of electrokinetic solution 120 from electrokinetic supply reservoir 110, through electrokinetic porous media 112, and into electrokinetic solution receiving chamber 114.

As electrokinetic solution receiving chamber 114 receives electrokinetic solution 120, movable partition 124 is forced to move in the direction of arrows A1. Such movement is evident by a comparison of FIG. 1 to FIG. 2. As movable partition 124 moves, infusion liquid 130 is displaced (i.e., “pumped”) out of infusion reservoir 126 through infusion reservoir outlet 128 in the direction of arrow A1. Electrokinetic engine 106 can continue to displace electrokinetic solution 120 until movable partition 124 reaches a predetermined point near infusion reservoir outlet 128, thereby displacing a predetermined amount (e.g., essentially all) of infusion liquid 130 from infusion reservoir 126.

It is evident from the description above and a comparison of FIGS. 1 and 2, that the second dispensing state represented by FIG. 2 is achieved by electrokinetically displacing (i.e., pumping or dispelling) a portion of infusion liquid that is present within infusion reservoir 126 in the first dispensing state represented by FIG. 1.

The rate of displacement of infusion liquid 130 from infusion reservoir 126 is directly proportional to the rate at which electrokinetic solution 120 is pumped from electrokinetic supply reservoir 110 to electrokinetic solution receiving chamber 114. The proportionality between the rate of displacement of the infusion liquid (such as an insulin containing infusion liquid) and the rate at which the electrokinetic solution is pumped can be, for example, in the range of 1:1 to 4:1. Furthermore, the rate at which electrokinetic solution 120 is pumped from electrokinetic supply reservoir 110 is a function of the voltage and current applied by first electrode 116 and second electrode 118 and various electro-physical properties of electrokinetic porous media 112 and electrokinetic solution 120 (such as, for example, zeta potential, permittivity of the electrokinetic solution and viscosity of the electrokinetic solution).

Further details regarding electrokinetic engines, including materials, designs, operation and methods of manufacturing, are included in U.S. patent application Ser. No. 10/322,083 filed on Dec. 17, 2002, which has been incorporated by reference. Other details are also discussed in U.S. patent application Ser. No. 11/112,867 filed on Apr. 21, 2005, which is hereby incorporated herein by reference in its entirety. More details are also disclosed in the U.S. patent application entitled “Electrokinetic Infusion Pump System” (Attorney Docket No. 106731-5), filed concurrently herewith. Although a particular electrokinetic engine is depicted in a simplified manner in FIGS. 1 and 2, any suitable electrokinetic engine can be employed in embodiments of the present invention including, but not limited to, the electrokinetic engines described in the aforementioned applications.

A position detector of an electrokinetic infusion pump 102 can be configured to sense (or determine) the position of movable partition 124. Based on the sensed position of movable partition 124 (as communicated by feedback signal FB), closed loop controller 104 can determine the dispensing state (e.g., the displacement position of movable partition 124 at any given time and/or as a function of time, the rate of displacement of infusion liquid 130 from infusion reservoir 126, and the rate at which electrokinetic solution 120 is pumped from electrokinetic supply reservoir 110 to electrokinetic solution receiving chamber 114).

Based on such a determination of dispensing state, closed loop controller 104 can control (i.e., can command and manage) the dispensing state by, for example, (i) adjusting the voltage and/or current applied between first electrode 116 and second electrode 118 or (ii) maintaining the voltage between first electrode 116 and second electrode 118 constant while adjusting the duration during which power is applied between the first electrode 116 and the second electrode 118. For example, by adjusting the voltage and/or current applied across first electrode 116 and second electrode 118, the rate at which electrokinetic solution 120 is displaced from electrokinetic supply reservoir 110 to electrokinetic solution receiving chamber 114 and, therefore, the rate, at which infusion liquid 130 is displaced through infusion reservoir outlet 128, can be accurately and beneficially controlled.

The closed loop control of electrokinetic infusion pumps described above beneficially compensates for variations that may cause inconsistent displacement (i.e., dispensing) of infusion liquid 130 including, but not limited to, variations in temperature, downstream resistance, occlusions and mechanical friction.

Electrokinetic supply reservoir 110 can be partially or wholly collapsible. For example, electrokinetic supply reservoir 110 can be configured as a collapsible sack. Such collapsibility provides for the volume of electrokinetic supply reservoir 110 to decrease as electrokinetic solution 120 is displaced therefrom. Such a collapsible electrokinetic supply reservoir can also serve to prevent formation of a vacuum within electrokinetic supply reservoir 110.

Infusion module housing 122 can be, for example, at least partially rigid to facilitate the movement of movable partition 124 and the reception of electrokinetic solution 120 pumped from electrokinetic supply reservoir 110.

Movable partition 124 is configured to prevent migration of electrokinetic solution 120 into infusion liquid 130, while minimizing resistance to its own movement (displacement) as electrokinetic solution receiving chamber 114 receives electrokinetic solution 120 pumped from electrokinetic supply reservoir 110. Movable partition 124 can, for example, include elastomeric seals that provide intimate, yet movable, contact between movable partition 124 and infusion module housing 122. In addition, movable partition 124 can have, for example, a piston-like configuration or be configured as a movable membrane and/or bellows.

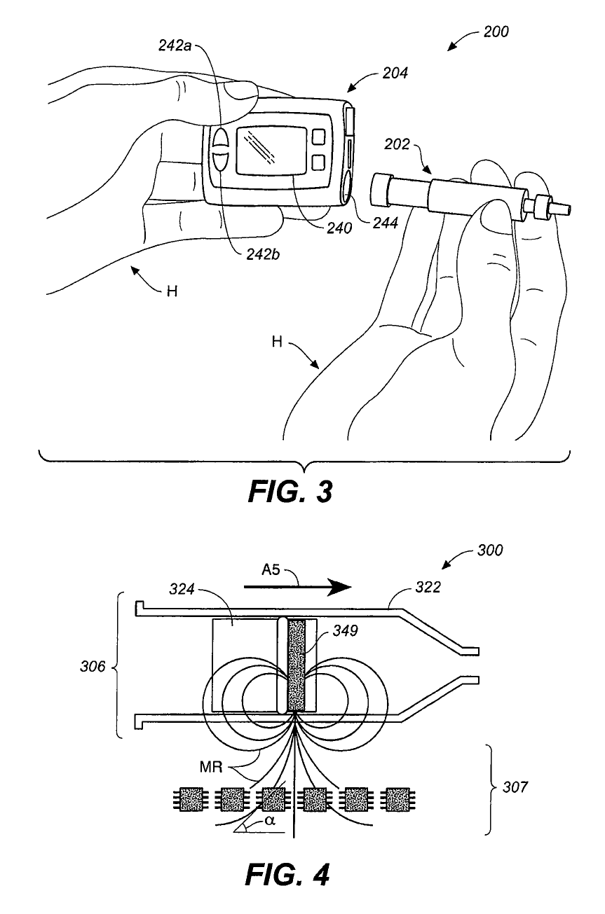

FIG. 3 is a simplified perspective illustration of an electrokinetic infusion pump system 200 according to another exemplary embodiment of the present invention being manipulated by a user's hands (H). Electrokinetic infusion pump system 200 includes an electrokinetic infusion pump 202 and a closed loop controller 204.

Electrokinetic infusion pump 202 and closed loop controller 204 can be handheld, and/or mounted to a user by way of clips, adhesives or non-adhesive removable fasteners. For example, electrokinetic infusion pump system 200 can be configured to be worn on a user's belt, thereby providing an ambulatory electrokinetic infusion pump system. In addition, closed loop controller 204 can be directly or wirelessly connected to a remote controller or other auxiliary equipment (not shown in FIG. 3) that provide analyte monitoring capabilities and/or additional data processing capabilities.

Although not necessarily depicted in FIG. 3, electrokinetic infusion pump 202 and closed loop controller 204 include components that are essentially equivalent to those described above with respect to electrokinetic infusion pump 102 and closed loop controller 104. In addition, closed loop controller 204 includes display 240, input keys 242a and 242b, and insertion port 244.

Display 240 can be configured, for example, to display a variety of information, including infusion rates, error messages and logbook information. During use of electrokinetic infusion pump system 200, and subsequent to electrokinetic infusion pump 202 having been filled with infusion liquid, electrokinetic infusion pump 202 is inserted into insertion port 244. Upon such insertion, operative electrical communication is established between closed loop controller 204 and electrokinetic infusion pump 202. Such electrical communication includes the ability for closed loop controller 204 to receive a feedback signal FB from an anisotropic magnetic resistive displacement position sensor of electrokinetic infusion pump 202 and operative electrical contact with first and second electrodes of electrokinetic infusion pump 202.

One skilled in the art will recognize that an infusion set (not shown but typically including, for example, a connector, tubing, needle and/or cannula and an adhesive patch) can be connected to the infusion reservoir outlet of electrokinetic infusion pump 202 and, thereafter, primed. As may be suitable for a particular infusion set, such attachment and priming can occur before or after electrokinetic infusion pump 202 is inserted into insertion port 244. After determining the position of a movable partition of electrokinetic infusion pump 202, voltage and current are applied across the electrokinetic porous media of electrokinetic infusion pump 202, thereby dispensing (pumping) infusion liquid.

Position Detectors

Various exemplary embodiments are directed to methods and systems for detecting the delivery of infusion liquids from an electrokinetic infusion pump. In particular embodiments, a position detector can be utilized to identify the delivery of the infusion liquid. Although many of the various position detectors described in the present application are described in the context of their use with electrokinetic engines, embodiments using other engines are also within the scope of embodiments of the present invention. Position detectors, as described in the present application, can be useful in many types of infusion pumps. These include pumps that use engines or driving mechanisms that generate pressure pulses in a hydraulic medium in contact with the moveable partition in order to induce partition movement. These driving mechanisms can be based on gas generation, thermal expansion/contraction, and expanding gels and polymers, used alone or in combination with electrokinetic engines. As well, engines in infusion pumps that utilize a moveable partition to drive delivery an infusion fluid (e.g., non-mechanically-driven partitions of an infusion pump such as hydraulically actuated partitions) can utilize a position detector to determine the location of the moveable partition.

One exemplary embodiment is drawn to a method of sensing fluid displacement in an infusion pump (e.g., an electrokinetic infusion pump). In particular, the infusion pump is actuated for moving a moveable partition to displace fluid from the pump. A position detector is utilized to detect the position of the moveable partition. The position of the moveable partition can be related to a quantity of fluid displaced from the pump. In another exemplary embodiment, a fluid delivery detector for an infusion pump includes a magnet coupled to a moveable partition of the pump. The position of the moveable partition can be correlated with an amount of fluid in the pump (e.g., infusion fluid) or amount of fluid located in a particular chamber of the pump (e.g., the amount of electrokinetic solution). One or more magnetic sensors can be located along a body of the infusion pump, such as along a length of conduit wall configured to hold infusion fluid or along a length of wall traveled by the moveable partition. A magnetic sensor can be configured to emit a signal when subjected to a magnetic field, for example a field generated by a magnet coupled to the moveable partition. The signal can be indicative of the position of the moveable partition.

Various type of hardware can be utilized as a position detector for an infusion pump. For example, optical components can be used to determine the position of a movable partition. Light emitters and photodetectors can be placed adjacent to an infusion housing, and the position of the movable partition determined by measuring variations in detected light. In other examples, a linear variable differential transformer (LVDT) can be used. When a LVDT is used, the moveable partition can include an armature made of magnetic material. A LVDT that is suitable for use in the present application can be purchased from RDP Electrosense Inc., of Pottstown, Pa.

In some embodiments, the position detector includes a magnetic sensor configured to detect the position of a moveable partition. For example, a movable partition can include a magnet, and a magnetic sensor can be used to determine the partition's position. The terms “magnetic sensor” and “magnetic position sensor” are used to refer to sensors that are generally capable of sensing a magnetic field. For example, the magnetic sensors can yield a signal representative of the direction of a magnetic field. Within the present application, specific examples of magnetic sensors include the use of a magnetorestrictive waveguide and an anisotropic magnetic resistive sensor. A variety of other magnetic sensors, including ones understood by those skilled in the art, can also be applied with the embodiments described herein (e.g., Hall-Effect sensors, magnetiresistive sensors, electronic compass units, etc.).

FIG. 10 illustrates the principles of one type of magnetic position sensor 176. Magnetic position sensor 176, suitable for use in this invention, can be purchased from MTS Systems Corporation, Sensors Division, of Cary, N.C. In magnetic position sensor 176, a sonic strain pulse is induced in magnetostrictive waveguide 177 by the momentary interaction of two magnetic fields. First magnetic field 178 is generated by movable permanent magnet 149 as it passes along the outside of magnetostrictive waveguide 177. Other types of magnets other than permanent magnets can also be utilized. Second magnetic field 180 is generated by current pulse 179 as it travels down magnetostrictive waveguide 177. The interaction of first magnetic field 178 and second magnetic field 180 creates a strain pulse. The strain pulse travels, at sonic speed, along magnetostrictive waveguide 177 until the strain pulse is detected by strain pulse detector 182. The position of movable permanent magnet 149 is determined by measuring the elapsed time between application of current pulse 179 and detection of the strain pulse at strain pulse detector 182. The elapsed time between application of current pulse 179 and arrival of the resulting strain pulse at strain pulse detector 182 can be correlated to the position of movable permanent magnet 149.

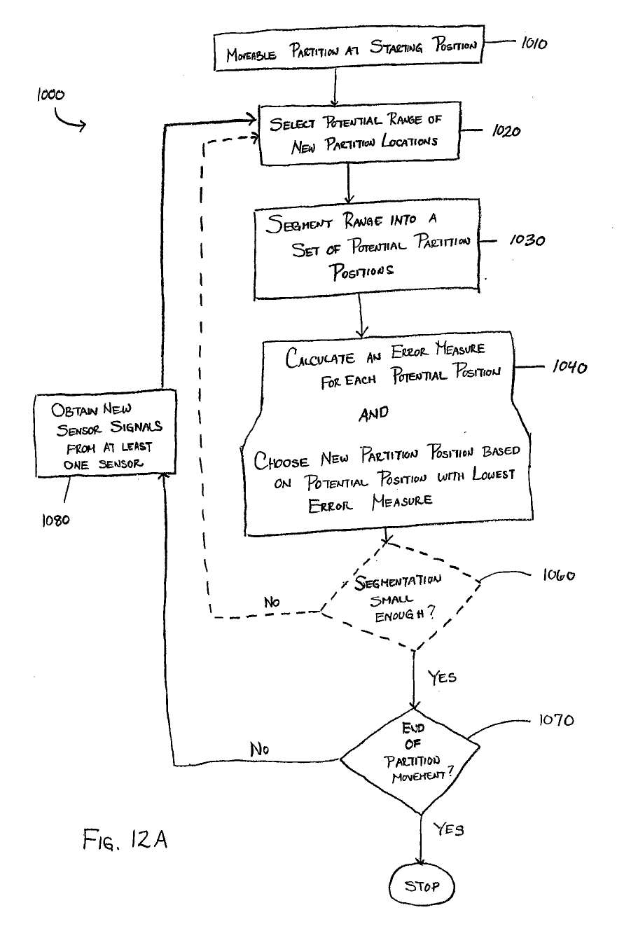

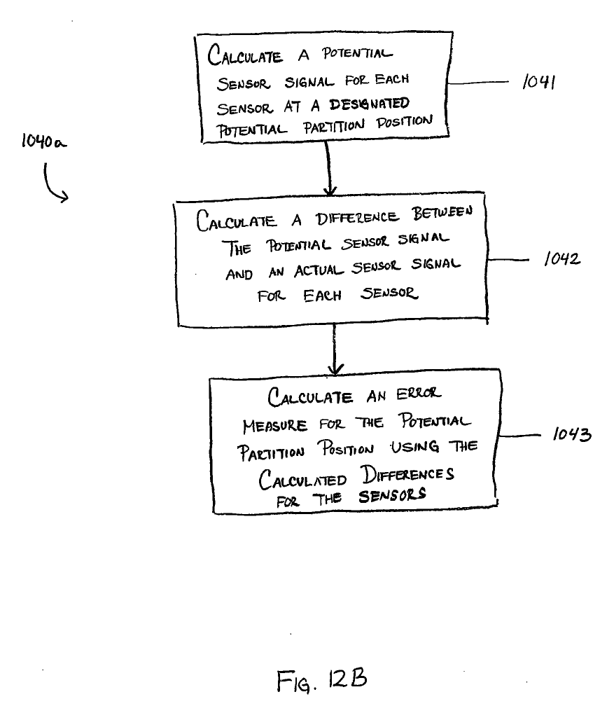

FIGS. 11A and 11B illustrate portions of an electrokinetic infusion pump utilizing a magnetic sensor of the type shown in FIG. 10, consistent with an embodiment of the present invention. FIGS. 11A and 11B include electrokinetic infusion pump 103, closed loop controller 105, magnetic position sensor 176, and position sensor control circuit 160. Position sensor control circuit 160 is connected to closed loop controller 105 by way of feedback 138. Electrokinetic infusion pump 103 includes infusion housing 116, electrokinetic supply reservoir 106, electrokinetic porous media 108, electrokinetic solution receiving chamber 118, infusion reservoir 122, and moveable partition 120. Moveable partition 120 includes first infusion seal 148, second infusion seal 150, and moveable permanent magnet 149. Infusion reservoir 122 is formed between moveable partition 120 and the tapered end of infusion housing 116. Electrokinetic supply reservoir 106, electrokinetic porous media 108, and electrokinetic solution receiving chamber 118 contain electrokinetic solution 114, while infusion reservoir 122 contains infusion liquid 124. Voltage is controlled by closed loop controller 105, and is applied across first electrode 110 and second electrode 112. Magnetic position sensor 176 includes magnetostrictive waveguide 177, position sensor control circuit 160, and strain pulse detector 182. Magnetostrictive waveguide 177 and strain pulse detector 182 are typically mounted on position sensor control circuit 160.

In FIG. 11A, moveable partition 120 is in first position 168. Position sensor control circuit 160 sends a current pulse down magnetostrictive waveguide 177, and by interaction of the magnetic field created by the current pulse with the magnetic field created by moveable permanent magnet 149, a strain pulse is generated and detected by strain pulse detector 182. First position 168 can be derived from the time between initiating the current pulse and detecting the strain pulse. In FIG. 11B, electrokinetic solution 114 has been pumped from electrokinetic supply reservoir 106 to electrokinetic solution receiving chamber 118, pushing moveable partition 120 toward second position 172. Position sensor control circuit 160 sends a current pulse down magnetostrictive waveguide 177, and by interaction of the magnetic field created by the current pulse with the magnetic field created by moveable permanent magnet 149, a strain pulse is generated and detected by strain pulse detector 182. Second position 172 can be derived from the time between initiating the current pulse and detecting the strain pulse. Change in position 170 can be determined using the difference between first position 168 and second position 172. As mentioned previously, the position of moveable partition 120 can be used in controlling flow in electrokinetic infusion pump 103.

Another type of magnetic sensor that can be utilized is an anisotropic magnetic resistive (AMR) displacement position sensor. AMR displacement position sensors are particularly beneficial for use in infusion pumps and infusion pump systems since they can be configured with a relatively large spacing between a magnet that interacts with the AMR displacement position sensor and the AMR displacement position sensor. Moreover, AMR displacement position sensors are relatively inexpensive and compatible with conventional printed circuit board (PCB) manufacturing techniques.

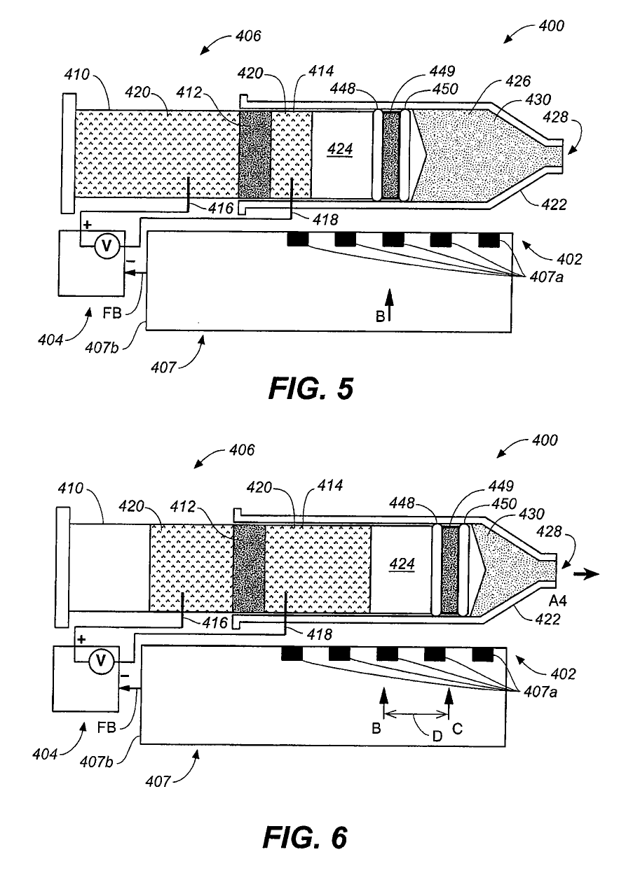

FIG. 4 is a simplified cross-sectional and schematic depiction of a portion of an electrokinetic infusion pump 300 according to a further exemplary embodiment of the present invention. Electrokinetic infusion pump 300 includes an integrated infusion module and electrokinetic engine 306 and an array of six AMR displacement position sensors 307 (that are in operative communication with a sensor measurement module (not depicted in FIG. 3) of electrokinetic infusion pump 300). The array of AMR displacement position sensors 307 is configured to sense a dispensing state of the integrated infusion module and electrokinetic engine 306. It should be noted that although, for clarity, FIG. 4 does not depict the sensor measurement module, such a sensor module is depicted and described with respect to FIGS. 5 and 6.

Integrated infusion module and electrokinetic engine 306 includes an infusion module housing 322 and a movable partition 324. Movable partition 324 includes a permanent magnet 349; other types of magnets can also be substituted. Integrated infusion module and electrokinetic engine 306 also includes components that are essentially identical to those described above with respect to the embodiment of FIGS. 1 and 2. However, for the sake of clarity, only those components relevant to the present discussion are depicted in FIG. 4.

Each individual AMR displacement position sensor in the array of AMR displacement position sensors 307 can be any suitable AMR displacement position sensor including, for example, AMR displacement position sensor HMC1501 and AMR displacement position sensor HMC1512 (commercially available from Honeywell Corporation, Solid State Electronics Center, of Plymouth, Minn., USA).

An AMR displacement position sensor typically includes a thin strip(s) of ferrous material (not depicted in FIG. 4). When an external magnetic field (MR) originating from permanent magnet 349 is applied to the thin strip of ferrous material, the resistance of the thin strip of ferrous material changes. The magnitude of the resistance change is a function of the angle between the external magnetic field (MR) and an axis of the thin strip of ferrous material (depicted as angle α in FIG. 4). This angle varies as permanent magnet moves past each of the individual AMR displacement sensors in the array of AMR displacement sensors 307. The individual AMR displacement sensors output a differential voltage signal that is indicative of the resistance and, thus, indicative of the angle and of the position of permanent magnet 349.

In the embodiment of FIG. 4, permanent magnet 349 is mounted to movable partition 324, and is disposed in close operative proximity (i.e., spacing or gap) to array of AMR displacement position sensors 307. The proximity of the movable partition 324 to AMR displacement position sensor 307 is dependent on the magnetic strength and dimensions of the permanent magnet but can be, for example, in the range of about 1 mm to about 12 mm. In general, it can be desirable to predetermine the magnetic strength of the permanent magnet such that the AMR displacement position sensors are saturated by the magnetic field. This can typically be achieved with, for example, an 80 Gauss magnetic field. In addition, the number of individual AMR displacement position sensors in the array can depend on the overall travel distance of the movable partition.

As movable partition 324 and movable permanent magnet 349 travel in the direction indicated by arrow A5, the angle between external magnetic field MR and each sensor in the array of AMR displacement position sensors 307 changes, causing a change in the resistance of a thin strip(s) of ferrous material inside each AMR displacement position sensor of the array.

Based on a differential output of each AMR displacement position sensor that is indicative of the resistance, the position of movable partition 324 and movable permanent magnet 349 can be determined, relative to the position of AMR displacement position sensor 307.

Although, for the purpose of explanation only, FIG. 4 depicts an array of six AMR displacement position sensors, any suitable number of AMR displacement sensors can be employed with the embodiments of the invention discussed herein—unless otherwise specifically stated. For example, a single AMR displacement position sensor can be employed if the distance traveled by a movable partition 324, and hence by a permanent magnet, is within the measurement range of such a single AMR displacement position sensor (e.g., the range being such that the AMR sensor can sense the location of a magnet to within a particular resolution error such as about 0.01 μm or about 1.0 μm or some other selected value). If the distance traveled by a movable partition and permanent magnet exceed the measurement range of a single AMR displacement position sensor, an array of multiple AMR displacement position sensors (such as that depicted in FIG. 4) can be employed. The number of position sensors utilized can be sufficient to span a selected distance such as the total distance potentially traveled by an infusion pump's moveable partition. For example, if R is a measurement distance range of one AMR sensor and L is the total length potentially traveled by a moveable partition, the total number of AMR sensors, N, can satisfy the relationship, NR≧L, to allow accurate identification of the location of the moveable partition.

FIG. 5 is a simplified cross-sectional depiction of an electrokinetic infusion pump system 400 according to a further exemplary embodiment of the present invention in a first dispense state, while FIG. 6 depicts electrokinetic infusion pump system 400 in a second dispense state.

Referring to FIGS. 5 and 6, electrokinetic infusion pump system 400 includes an electrokinetic infusion pump 402 and a closed loop controller 404. As will be clear to one skilled in the art from the following description, electrokinetic infusion pump 402 includes an integrated infusion module and electrokinetic engine (collectively element 406) and an AMR displacement position sensor 407. Moreover, AMR displacement position sensor 407 includes an array of five AMR sensors 407a and a sensor measurement module 407b. In the embodiment of FIGS. 5 and 6, sensor measurement module 407b is configured to receive signals from the five AMR sensors 407a (e.g., the aforementioned differential voltage signals), interpret the received signals and convert the interpreted signals to a digital signal (i.e., a digital FB signal) that is correlated to the position of the permanent magnet. However, once apprised of the present disclosure one skilled in the art can readily devise other suitable configurations for a sensor measurement module employed with embodiments of the present invention.

Integrated infusion module and electrokinetic engine 406 includes an electrokinetic supply reservoir 410, electrokinetic porous media 412, electrokinetic solution receiving chamber 414, first electrode 416, second electrode 418, and electrokinetic solution 420 (depicted as upwardly pointing chevrons). Integrated infusion module and electrokinetic engine 406 also includes infusion module housing 422, movable partition 424, infusion reservoir 426, infusion reservoir outlet 428 and infusion liquid 430 (depicted as dotted shading).

Movable partition 424 includes a first infusion seal 448, a permanent magnet 449 and second infusion seal 450. Permanent magnet 449 of movable partition 424 is at position B in the first dispense state of FIG. 5 and at position C in the second dispense state of FIG. 6 (with the movement between positions B and C indicated by arrow A4 of FIG. 5). The distance between position B and position C is labeled D in FIG. 6.

Sensor measurement module 407b can be configured to provide a feedback signal FB to closed loop controller 404, from which the position of movable partition 424 and the dispense state of electrokinetic infusion pump system 400 can be derived.

In some embodiments, a sensor measurement module 407b, as exemplified in FIGS. 5 and 6, can include, or be configured as, a temperature signal compensator. A temperature signal compensator can be configured to receive signals from a position detector (e.g., one or more AMR displacement sensors 407a) and a temperature signal from a temperature sensor (not shown) so as to produce a temperature-corrected signal indicative of the position of the moveable partition. Such embodiments can help reduce errors produced by position detectors that are subjected to varying temperature environments.

A variety of temperature sensors can be utilized (e.g., a thermocouple or a Pt resistor), and oriented to provide an accurate temperature reading of the environment of the position detector. The temperature sensor can be integrated into the sensor measurement module, or be a remotely connected unit. The temperature signal compensator can apply information that adjusts the signal received by a position detector to account for signal attenuation due to the temperature of the detector. For example, the temperature dependence of an AMR sensor can be characterized by a look-up table of data, or coefficients of a polynomial or other mathematical function, which is a function of temperature, the data being obtained, for example, by calibrating the performance of the detector at varying temperatures. Such data can be stored within the compensator or in a separately connected unit. Depending upon the temperature detected, the compensator can utilize the data to adjust a received signal and produce a subsequent signal that compensates for the detected temperature.

Those skilled in the art will appreciate that a number of other techniques can be used to produce the data needed to alter a detector signal to account for temperature variations. As well, though temperature compensation for position detectors is discussed herein with respect to the use of a temperature signal compensator, other types of hardware implementation can also be utilized to carry out the functionality described by the compensator. Indeed, such functionality provides methods consistent with embodiments of the invention. Such methods can include some or all of the functionality described herein. All these variations are within the scope of the present application.

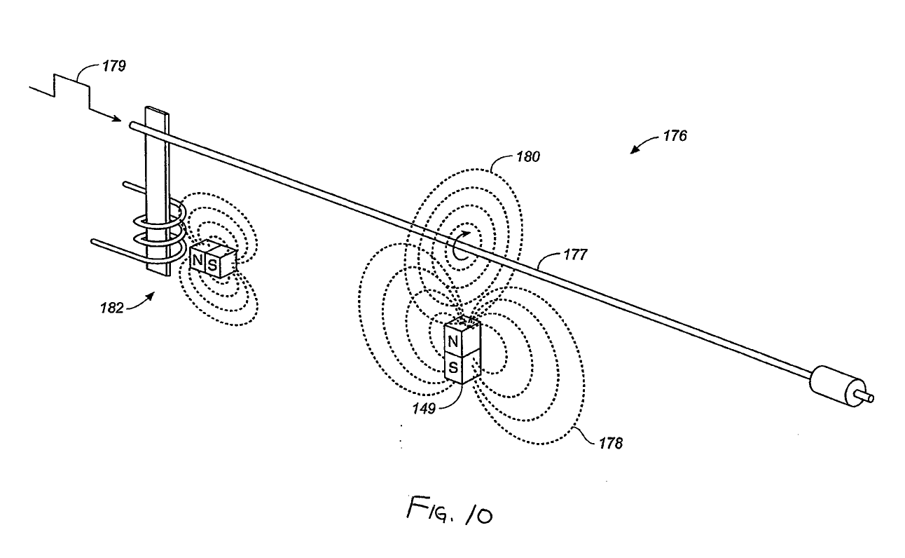

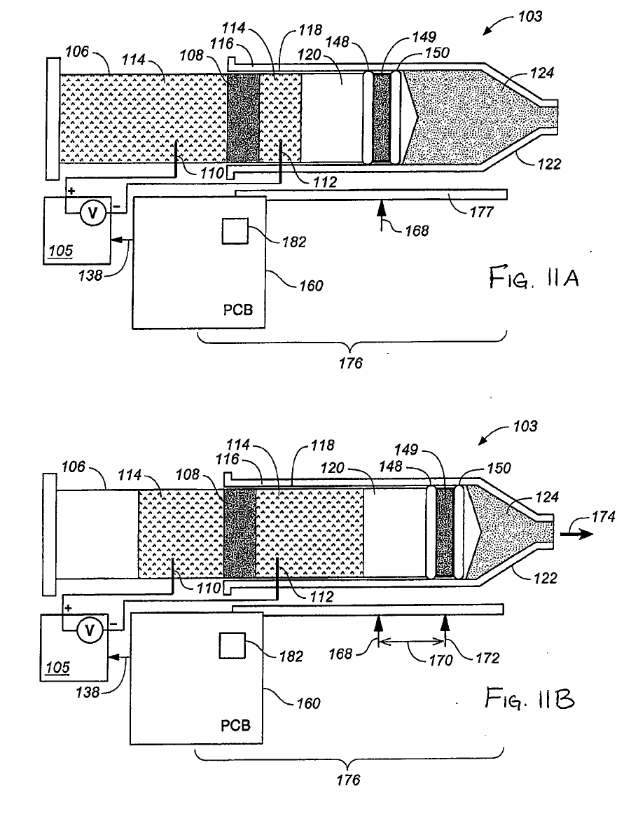

FIG. 9 is a flow diagram illustrating a method 800 for the closed loop control of an electrokinetic infusion pump according to an embodiment of the present invention. Method 800 includes, at step 810, sensing a dispensing state of an electrokinetic infusion pump with an AMR displacement position sensor. The AMR displacement position sensor and electrokinetic infusion pump can be any such sensor and electrokinetic infusion pump as described herein with respect to embodiments of the present invention.

Subsequently, the sensed dispensing state of the electrokinetic infusion pump is signaled to a closed loop controller via a feedback signal, as set forth in step 820. The closed loop controller then determines the dispensing state of the electrokinetic infusion pump based on the feedback signal, as set forth in step 830.

Subsequently, at step 840, the dispensing state of the electrokinetic infusion pump (e.g., infusion liquid displacement rate) is controlled by the closed loop controller by the sending command signals from the closed loop controller to an electrokinetic engine of the electrokinetic infusion pump. Method 800 can be practiced using electrokinetic infusion pump systems according to the present invention including the embodiments of FIGS. 1 through 8. Further details regarding closed loop control schemes that can be utilized with embodiments of the present invention are presented in the copending U.S. patent application entitled “Infusion Pump with Closed Loop Control and Algorithm” (Attorney Docket No. 106731-3), which is concurrently filed with the present application and incorporated herein by reference in its entirety.

Electrokinetic infusion pumps, electrokinetic infusion pump systems and associated methods according to embodiments of the present invention can provide for beneficially accurate determination of dispensing states. Moreover, the AMR displacement position sensors employed do not require any direct electrical connection to the electrokinetic infusion pump or electrokinetic engine since they sense displacement position via a magnetic field.

Identifying the Location of a Moveable Partition with a Position Detector

Though the signal produced by a position sensor can be mapped to a particular position of a moveable partition of an infusion pump, such a mapping can be labor intensive. For instance, if the sensor signal output is non-linear with respect to the position of the moveable partition, the mapping between sensor signal output to position can require substantial computational effort. As an example, if a moveable partition is designed to travel a length of 25 millimeters and the resolution of the partition position is desired to within about a micron, potentially 25,000 search iterations can be required to determine the position associated with a particular sensor signal. Furthermore, if multiple position sensors are utilized, the number of iterations can be multiplied by the number of sensors used. The substantial computational effort required to process so many iterations can slow signal processing, and ultimately hinder other processes such as closed loop control of fluid displacement from the infusion pump. Accordingly, a need exists for faster and/or computationally simpler methods and systems for determining the position of moveable partition to a desired degree of linear resolution.

Some embodiments herein are directed toward systems and methods of locating a position of a moveable partition in an infusion pump using one or more displacement sensors. As previously indicated herein, when a moveable partition is used to induce liquid movement in an infusion pump, the position and relative movement of the partition can be used to determine an amount of fluid that is displaced. Accordingly, the methods described herein can also be used to determine fluid displacement from an infusion pump. Such methods can also be used to provide a position of the moveable partition to a closed loop control algorithm, which can control subsequent fluid delivery from an infusion pump. Furthermore, the methods described herein can be applicable to a variety of types of infusion pumps including electrokinetic infusion pumps among others that utilize a moveable partition to drive fluids such as infusion fluid. As well, the types of position sensors that can be utilized can also vary, and include the kinds of sensors previously described herein. In particular embodiments, the sensor can provide a signal based at least in part on an actual position of the moveable partition, a signal based at least in part on a detected magnetic field, and/or the sensor can include one or more AMR displacement position sensors (e.g., at least two position sensors).

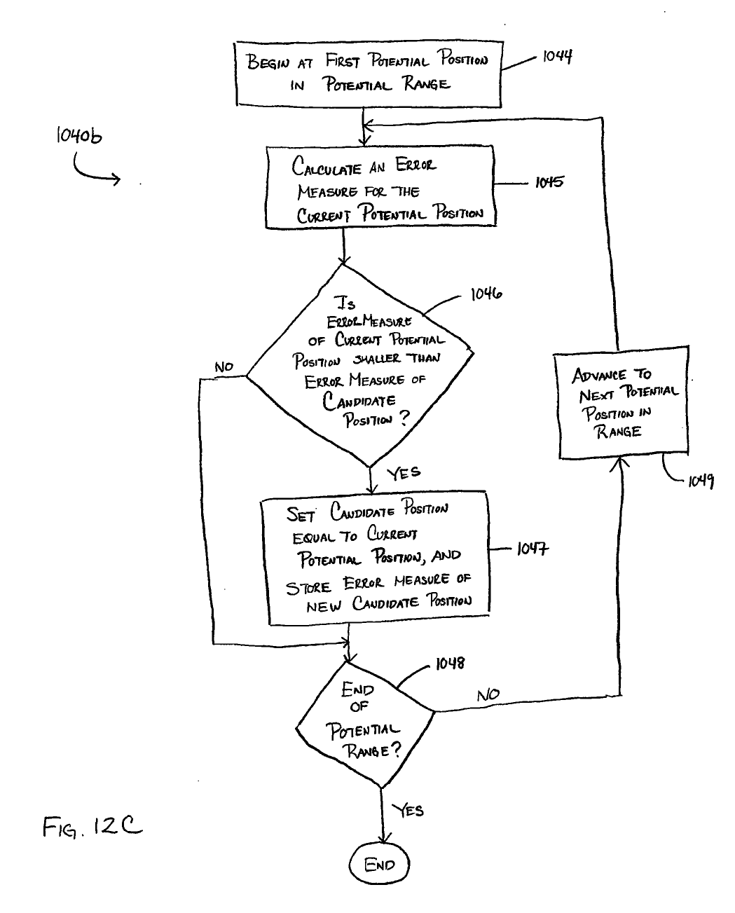

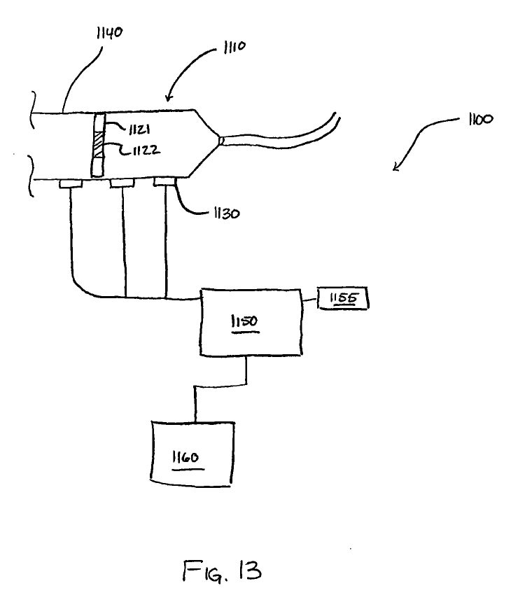

FIG. 12A presents a flow chart corresponding to a method for locating a position of a moveable partition of an infusion pump in accord with an exemplary embodiment. The infusion pump can include at least one displacement sensor, which can be configured to produce a signal indicating the position of the moveable partition. The method 1000 begins by identifying the starting position of a moveable partition 1010. The starting position can be anywhere where that the partition can be located such as the position when the infusion pump has a full capacity of infusion fluid stored therein. As the moveable partition proceeds through the infusion pump, the position of the partition can be identified using the following steps. A potential range of new partition positions is identified 1020. The potential range can be segmented into a set of potential partition positions 1030, which can span the potential range. A error measure can be calculated for each of the new potential partition positions, and a new partition position selected from the new potential partition positions based upon the position having the lowest calculated error measure 1040. Steps 1010, 1020, 1030, and 1040 can be repeated according to an operational mode of the infusion pump. For example, if the moveable partition has not reached a selected end position 1070, new sensor signals can be collected from one or more of the displacement sensors 1080, followed by repetition of steps 1010, 1020, 1030, and 1040. When a selected end position has been reached, the steps of the method can be halted. Of course, other indicators can also be utilized to halt continuous detection of the partition's position (e.g., non functioning of the pump, or user initiated stoppage).

By utilizing particular methodologies, such as those described herein, for selecting the potential range of partition locations and for segmenting the potential range, an expedited identification of a new partition position can be achieved having a selected degree of accuracy relative to former techniques that required investigating the entire range of movement of a moveable partition with a degree of accuracy necessitating a large number of calculations. In particular, the method exemplified by the flow chart of FIG. 12A can reduce the number of calculations required to obtain the new partition position within a selected degree of accuracy.

For example, simulated mathematical calculations were performed based upon the techniques described herein. A total of four sensors were coupled to a microcontroller MSP430F1611 (Texas Instruments Incorporated, Dallas, Tex.) running at 8 MHz, and used to output a value representing the location of a magnet. When the microcontroller utilized the algorithm discussed herein, the technique reduced the time for finding a new partition position from a time of approximately one minute to a time of about 215 milliseconds.

Selection of a potential range of new partition locations 1020 can be determined in a variety of manners. For example, the potential range can be the entire potential range that a moveable partition can travel. In some instances a subset of the entire potential range can be chosen. Such a subset can be determined using numerous criteria such as the last calculated location of the moveable partition, the number of position sensor used, the location of one or more of the position sensors, and/or some range selected by a user or manufacturer. In one example, the range can be designated by the last calculated or known position of the moveable partition±a selected half-range value. The selected half-range value can be chosen based on a convenient scale (e.g., a half, a quarter, or some other fraction of the total potential partition travel length), and/or can be based upon some algorithm to help provide successively smaller ranges to investigate, as discussed more in depth herein. In another example, a range can be selected from a set of potential ranges, each potential range being 1/N times the total potential partition travel length, where N is the number of position sensors utilized. The particular potential range can be selected based at least in part upon the previously calculated or known partition position. For instance, if a potential travel length of 24 mm is available for a moveable partition and four AMR position sensors are used, the potential ranges can be 0-6 mm, 6-12 mm, 12-18 mm, and 18-24 mm. Accordingly, if the last known position of the partition is 8.05 mm, the range of 6-12 mm can be selected. Those skilled in the art will appreciate that a number of other methods can also be utilized to select a potential range, in accord with embodiments of the invention discussed herein (e.g., the number of potential ranges need not be equal to the number of sensors utilized).

Segmenting a potential range into a set of potential partition positions 1020 can be achieved to enable quick and accurate assessment of a partition's position. In some instances, the set of potential partition positions can be equally spaced apart, though this is not required. In particular, the step size between the potential partition positions in the range can be chosen using a number of criteria. For example, the step size can be of the order of the resolution desired for knowing the partition's position (e.g., knowing the position to within at least about a micron, or a tenth of a micron, or a hundredth of a micron). In another example, the step size can be substantially larger than the desired resolution to facilitate a rapid coarse evaluation of the position of the partition. Subsequent sequential determinations of the partition's position can utilize successively smaller step sizes. This choice can be coordinated with the choice of potential range, and is discussed more in depth herein.

In step 1040 of the method 1000, an error measure is calculated for each potential position in the potential range. An error measure can be calculated based at least in part upon one or more actual displacement sensor signals obtained from one or more of the position sensors. In one embodiment, an error measure can be a measure of the difference between an actual sensor displacement signal and a predicted displacement sensor signal for one or more position sensors at the designated potential position. In one example, the exact difference between an actual displacement sensor signal of a sensor and a predicted displacement sensor signal based upon a model using potential position as an input to produce the predicted signal is utilized. Other measures of difference can also be used such as the square of the difference between an actual sensor signal and a predicted sensor signal or the absolute value of the difference.

The calculation of an error measure 1040a for each potential position in a potential range can be performed according to the steps of a method shown by the flow chart of FIG. 12B in accord with an embodiment of the invention. A potential sensor signal for each position sensor at a designated potential partition position is calculated 1041. In general, the potential sensor signals are obtained using some predictive model of sensor behavior for each of the sensor. For example, each sensor can be calibrated to determine what signal is generated depending upon the particular position of a partition in an infusion pump. Such calibration data can be stored in a look-up table format of the memory of a processor for later recall. In another instance, a mathematical function can be created, such as a fitted polynomial, and stored in a memory of a processor. Accordingly, by identifying a particular partition position, the function can be used to generate a predicted sensor signal associated with that particular position. In a particular instance, a sixth order polynomial can be utilized as a model for each sensor. The predicted sensor signal can be generated by a microprocessor using the following formula:
yᵢ=x*(x*(x*(x*(x*(aᵢx+bᵢ)+cᵢ)+dᵢ)+eᵢ)+fᵢ)+gᵢ
where aᵢ, bᵢ, cᵢ, dᵢ, eᵢ, fᵢ, and gᵢ are the coefficients of the polynomial for the i^{th} sensor, x is the designated potential partition position, and yᵢ is the predicted sensor signal for the i^{th} sensor. Using the above formula allows a processor to only store six coefficients to hold the data necessary to predict the sensor signals. As well, the above form of the 6^{th} order polynomial reduces the number of multiplications required to obtain the predicted sensor signal from 11 to 6, relative to the typical polynomial form. Those skilled in the art will appreciate that many other methods of predicting a sensor signal can also be utilized within the scope of the present application (e.g., using other mathematical models or formulas, or stored look-up tables).

After obtaining the potential sensor signal for each sensor, a difference can be calculated between the potential sensor signal and an actual sensor signal for each sensor 1042. Such a difference can provide a measure of the deviation of the actual position of a moveable partition from the potential partition position used to calculate the potential sensor signal. It is expected that the difference in actual and predicted sensor signal should grow as the deviation between the actual and potential partition position grows.

The calculated difference between the potential and actual sensor signals for each sensor can be used to calculate the error measure 1043. The error measure can provide a convenient form for utilizing the calculated differences of step 1042 to provide a composite measure of the deviation of the actual partition position from the potential partition position used to calculate the predicted sensor signal. As previously noted, the error measure can simply be set equal to the difference between the actual and predicted sensor signals, in the case where only one sensor is utilized. When multiple sensors are utilized, it can be convenient to combine the differences for each of the sensors. For example, the error measure can be the sum of the squared differences for all the sensors, that is:

$$EM = {\sum }_{i} {( {A}_{i} - {P}_{i} )}^{2}$$

where EM is the error measure, Aᵢ is the actual sensor signal of the i^{th} sensor, and Pᵢ is the predicted sensor signal of the i^{th} sensor at a designated potential position. In another example, the error measure can be the sum of the absolute values of the differences for all the sensors, that is:

$$EM = {\sum }_{i} \vert {A}_{i} - {P}_{i} \vert$$

When an infusion pump utilizes multiple sensors, an error measure does not necessarily require combining actual and predicted sensor signal differences from all the sensors. In some embodiments, a subset of the sensors can be utilized in the calculation. The subset of sensors can be chosen on the basis of a variety of criteria, such as only utilizing those sensors whose measurement ranges include the last calculated partition position. In another example, only the two displacement sensor closest to the last calculated partition position are utilized; this can reduce potential sensor interference (with external magnetic fields) that may exist when a large number of sensors are used in an infusion pump. Those skilled in the art will appreciate that other techniques of calculating error measures can also be utilized consistent with embodiments of the invention, and all such embodiments are within the scope of the present application.

Referring back to the flow chart of FIG. 12A, the error measure for each of the potential positions can be used to choose a new partition position 1040. In particular, the new partition position can be set equal to the potential position having the lowest error measure. The determination of the potential position having the lowest error measure can be carried out using various techniques. One particular technique 1040b of carrying out step 1040 of FIG. 12A is depicted by the flow chart shown in FIG. 12C. First the current potential position can be set equal to the potential position corresponding to the beginning potential position in the selected potential range 1044. An error measure can then be calculated at the current potential position 1045 in accord with any of the techniques described within the present application. The calculated error measure associated with the current potential position is compared with an error measure associated with a candidate position 1046. If the error measure of the current potential position is smaller than the error measure of the candidate position, the candidate position can be assigned a new value equal to the current potential position, and its associated error measure can be stored 1047. If the error measure of the current potential position is greater than the error measure of the candidate position, step 47 can be omitted. If the current potential position is the last potential position in the potential range 1048, the new partition position can be assigned a value equal to the candidate position. Otherwise, the current potential position can be assigned a new value equal to the next potential position in the range 1049, and steps 1045, 1046, 1047, and 1048 are repeated. The technique 1040b can reduce the storage requirements necessary for searching for the lowest error measure among all the potential positions in a potential range since not all the error measures need be calculated and stored before searching for the lowest value. Other techniques for carrying out step 1040 of FIG. 12A can also be utilized, including calculating all the error measures for all potential positions before using a search technique to identify the lowest error measure in the assembled calculations.

The embodiment of locating a position of a moveable partition of an infusion pump depicted in FIG. 12A can optionally include a technique for expediting the search for a new partition position within a given resolution scale. In a particular instance, steps 1020, 1030, and 1040 can be repeated using a different potential range and/or a different segmentation of the range for each repetition of the steps. For example, given a new partition position and new sensor signals, from one or more sensors, after partition movement 1080, step 1020 can be performed by using a relatively large initial half-range (e.g., 1 mm) such that the range is the previous partition position ± the half range, that is the initial half-range is chosen to be large enough that the new partition position is very likely to be within the initial range.

Step 1030 is then performed by segmenting the range into a selected number of discrete potential positions. The selected number of potential positions can be chosen to correspond to a length that is substantially larger than the ultimate resolution of the potential position sought; this is to provide a coarse estimate of the location of the moveable partition. For example, in conjunction with the initial range, the spacing between the potential positions can be a particular fraction of the initial half-range (e.g., 0.1 mm).

Step 1040 can then be performed, utilizing any of the embodiments and techniques discussed herein, with the range and segmentation identified by steps 1020 and 1030.

Next, a check can be made to identify if the length corresponding to the segmentation performed in step 1030 is small enough, e.g., the length is of the resolution ultimately desired for identifying the partition position.

If the length is still too large, steps 1020, 1030, and 1040 can be repeated using the newly identified partition position of step 1040 and the previously obtained sensor signals. It can be advantageous to reduce either the potential range of new partition positions or the segmentation length in the subsequent repetition of steps 1020, 1030, and 1040. It can be especially advantageous to reduce both the size of the range and the segmentation length to provide a more accurate determination of the partition position while searching a smaller range. The steps 1020, 1030, and 1040 can be successively repeated until a segmentation length that is small enough is utilized.

The choice of a new range and new segmentation length can be by a variety of methods. In some instances, the new range can use a half-range from the new partition position that is some selected fraction of the previously utilized half-range, such as a fraction smaller than about ½, ¼, or a tenth of the previously utilized half-range. Accordingly, the new half-range can also be designated as a reduced factor of the previously utilized half-range (e.g., at least a factor of two, four, or 10). The choice of a new segmentation length can also be based upon some selected fraction of a previously utilized segmentation length (e.g., a fraction smaller than about ½, ¼, or a tenth of the previously utilized segmentation length). In some instances, both the half-range and the segmentation length can be reduced by an equal selected factor (e.g., reducing both the half-range and the segmentation length by a factor of at least 10 for each successive performance of steps 1020, 1030, and 1040). Those skilled in the art will recognize that a number of other ways of methodologies for reducing either, or both, the range and the segmentation length can be applied consistent with the scope of the present application.

Other embodiments of the invention are directed to systems and apparatus that can carry out the methods and techniques of locating a position of a moveable partition previously described, or portions of such methods and techniques. In one embodiment, illustrated in FIG. 13, a system 1100 for locating a moveable partition's position in an infusion pump 1110 includes a magnet 1121 coupled to a moveable partition 1122 and at least one sensor 1130 (e.g., magnetic sensor) coupled to a body 1140 of the infusion pump 1110. Each sensor 1130 can be configured to emit a signal when the sensor 1130 is subjected to a magnetic field of the magnet 1122. The system 1100 can further include a processor 1150 coupled to each of the sensors 1130. The processor 1150 can be configured to perform any number of the steps of the methods and techniques disclosed herein for identifying the position of the moveable partition. For example, the processor can be configured to identify a moveable partition's position by calculate a set of error measurements over a potential range of positions. The set of error measurements can depend at least in part upon at least one actual sensor measurement and a set of potential positions within the potential range. Error measurements, the potential range of positions, and actual sensor measurements (i.e., sensor signals) can be obtained in accordance with the techniques discussed herein. The system 1100 can further include other hardware to achieve the desired functionalities, such as a memory 1155 configured to store data associated with a potential sensor signal that can be used when calculating one or more error measures. The system 1100 can also include a closed loop controller 1160 coupled to the processor 1150 for controlling fluid delivery from the infusion pump 1110, in accordance with any of the embodiments discussed in the present application.

The various functionalities described with respect to the methods illustrated in FIGS. 12A-12C, and the system illustrated by FIG. 13, are all exemplary embodiments. It is understood that many variations of such methods and systems can be practiced within the scope of the present application. For example, the steps of the methods need not necessarily follow the exact order discussed herein. As well, the selected functionalities can be chosen and ordered to produce other embodiments of the invention beyond those described explicitly. All these variations are intended to be within the scope of the present disclosure.

It should be understood that various alternatives to the embodiments of the invention described herein may be employed in practicing the invention. It is intended that the following claims define the scope of the invention and that structures within the scope of these claims and their equivalents be covered thereby.

### EXAMPLE

The following example is provided to illustrate some aspects of the present application. The example, however, is not intended to limit the scope of any embodiment of the invention.

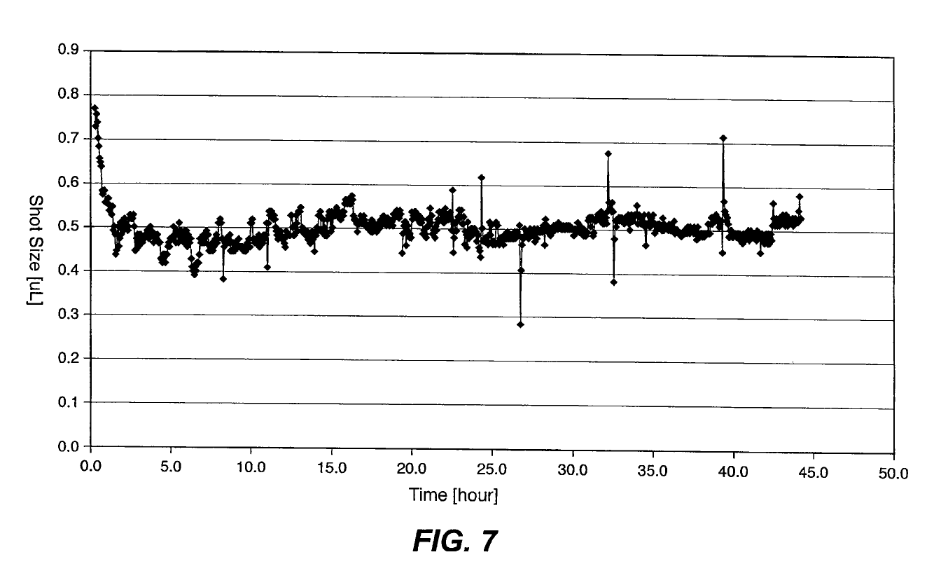

An experimental electrokinetic infusion pump system similar to those depicted in FIGS. 1, 2, 5 and 6 was employed to measure accuracy of infusion liquid dispensing under conditions of controlled temperature (±1° C.) and minimal vibration. FIG. 7 is a graph of shot size (i.e., the volume of infusion liquid dispensed during a given pumping cycle of 180 seconds) versus time obtained using this experimental system. During the collection of the data of FIG. 7, the electrokinetic engine of the experimental electrokinetic infusion pump system was controlled based on feedback signals received from the AMR displacement position sensor of the experimental electrokinetic infusion pump system. In particular, the portion of a pump cycle during which the electrokinetic engine was driven with an applied voltage of 75V was adjusted to target a shot size of 0.5 uL. The first nine points of FIG. 7 depict the adjust of shot size to the target of 0.5 uL by the closed loop controller of the experimental electrokinetic infusion pump system.

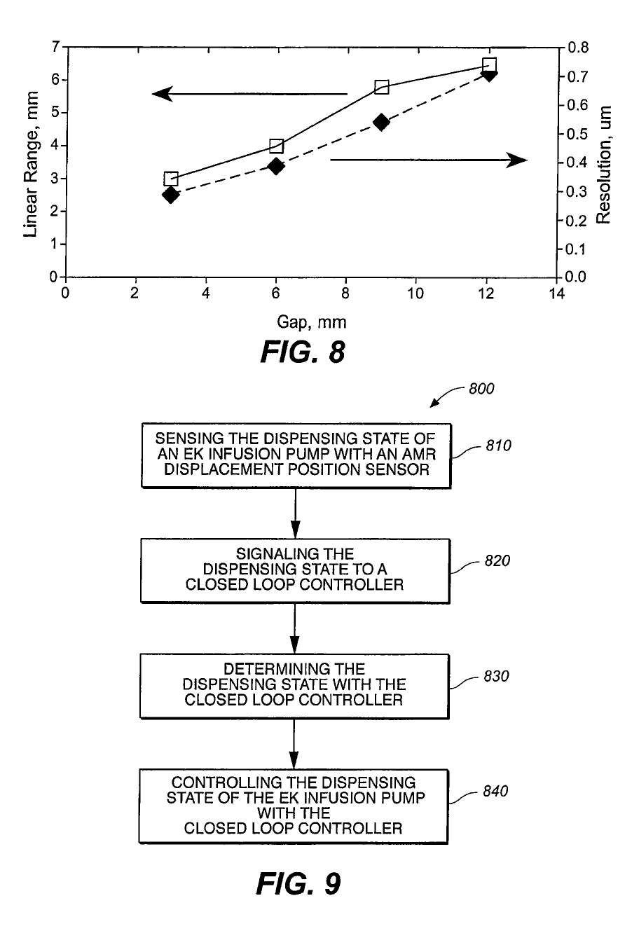

FIG. 8 is a graph of the linear range of movable partition movement and the measurement resolution versus gap for another experimental electrokinetic infusion pump according to the present invention. In this regard, the term “gap” refers to a distance between the permanent magnet of the movable partition and a single Honeywell HMC1501 AMR displacement position sensor. The data of FIG. 8 indicate that the measurement resolution is less than 1 um for gaps as large as 12 mm and that a linear range of 6.5 mm can be sensed with a gap of 12 mm.

What is claimed is:

1. A method of locating a position of a moveable partition for an infusion pump using at least one displacement sensor, comprising:
a) selecting a potential range of positions for the moveable partition of the infusion pump;
b) segmenting the potential range into a set of potential positions; and
c) selecting a new position for the moveable partition to correspond with the potential position having a lowest calculated error measure in a calculated set of error measures, each error measure corresponding to one potential position in the potential range, each error measure based at least in part upon an actual displacement sensor signal from each of the at least one displacement sensor and the potential position corresponding with the calculated error measure.

2. The method of claim 1, further comprising:
d) determining an amount of fluid displaced from the infusion based upon the new position of the moveable partition and a previous position of the moveable partition.

3. The method of claim 1, wherein the at least one displacement sensor provides the actual displacement sensor signal based at least in part upon an actual position of the moveable partition.

4. The method of claim 3, wherein the at least one displacement sensor provides the actual displacement sensor signal based at least in part upon a detected magnetic field.

5. The method of claim 4, wherein the at least one displacement sensor includes at least one anisotropic magnetic resistive sensor.

6. The method of claim 1, wherein the at least one displacement sensor comprises at least two displacement sensors.

7. The method of claim 6, wherein less than all of the plurality of displacement sensors are utilized in performing the method.

8. The method of claim 7, wherein only two displacement sensors located closest to the moveable partition are utilized in performing the method.

9. The method of claim 1, wherein selecting the potential range of positions includes using a last designated position of the moveable partition to select the potential range.

10. The method of claim 9, wherein selecting the potential range of positions includes selecting the range to be a selected distance before and after the last designated position of the moveable partition.

11. The method of claim 1, wherein selecting the new position includes calculating a measure of a difference between the actual displacement sensor signal and a predicted displacement sensor signal for at least one potential position in the range to determine at least one of the error measures.

12. The method of claim 11, wherein selecting the new position includes using a mean square error for each of the error measures.

13. The method of claim 12, wherein each mean square error corresponding to the one potential position is identified by
(i) calculating a difference between the actual displacement sensor signal and a predicted displacement sensor signal for each of the at least one displacement sensor, the predicted displacement sensor signal depending at least in part on the one potential position; and
(ii) calculating a mean square error by summing the squares of the calculated differences from each of the at least one displacement sensor at the one potential position.

14. The method of claim 11, wherein the predicted displacement sensor signal is provided by a calibrated model for each of the at least one displacement sensor.

15. The method of claim 14, wherein the calibrated model is a fitted polynomial.

16. The method of claim 1, wherein segmenting the potential range includes providing a set of equally spaced potential positions.

17. The method of claim 1, wherein steps a), b), and c) are repeated as the moveable partition proceeds through the infusion pump.

18. The method of claim 1, wherein steps a), b), and c) are repeated a plurality of times for a set of actual sensor signals taken from the at least one displacement sensor at a particular instance, each successive repetition of steps segmenting a corresponding potential range of positions for the moveable partition into equally spaced potential positions that are closer together, the corresponding potential range becoming smaller with each successive repetition of steps.

19. The method of claim 18, wherein for each successive repetition of steps a), b), and c) the corresponding potential range is reduced by at least a factor of two.

20. The method of claim 18, wherein for each successive repetition of steps a), b), and c) a segmentation spacing between the potential positions is reduced by at least a factor of two.

21. The method of claim 1, wherein step c) comprises:
(i) calculating the error measure at a current potential position of the moveable partition;
(ii) setting a candidate position of the moveable partition equal to either the current potential position or a previously calculated potential position based upon the error measures corresponding with the potential positions;
(iii) repeating steps (i) and (ii) for each of the potential positions in the range; and
(iv) setting the new position of the moveable partition equal to a last candidate position.

22. The method of claim 1, further comprising:
using the new position of the moveable partition in a closed loop control algorithm to control subsequent fluid delivery from the infusion pump.

23. The method of claim 1, wherein the infusion pump is an electrokinetic infusion pump.

24. A system for locating a position of a moveable partition in an infusion pump, comprising:
a magnet coupled to the moveable partition;
at least one magnetic sensor coupled to a body of the infusion pump, each of the at least one magnetic sensor configured to emit a signal when subjected to a magnetic field of the magnet; and
a processor coupled to each of the at least one magnetic sensor, the processor configured to identify the position of the moveable partition at least in part by calculating a set of error measurements over a potential range of positions, the set of error measurements depending in part upon at least one actual sensor measurement and a set of potential positions within the potential range.

25. The system of claim 24, wherein at least one magnetic sensor includes at least two magnetic sensors disposed along a distance traversable by the moveable partition.

26. The system of claim 24, wherein the at least one magnetic sensor includes at least one anisotropic magnetic resistive sensor.

27. The system of claim 24, wherein the processor is configured to identify the position of the moveable partition by equating the position with a corresponding potential position having a lowest error measurement.

28. The system of claim 27, wherein the processor is configured to calculate a set of error measurements by calculating a measure of a difference between an actual displacement sensor signal and a predicted sensor signal for each of the at least one magnetic sensor at each of the potential positions.

29. The system of claim 28, wherein the processor is configured to produce the predicted sensor signal based upon a predetermined model for each of the at least one magnetic sensors.

30. The system of claim 29, wherein the processor includes a memory configured to store the coefficients of a polynomial used as the model for the predicted sensor signals.

31. The system of claim 24, wherein the processor includes a memory configured to store at least one of the set of error measurements, each error measurement associated with a potential position.

32. The system of claim 24, further comprising:
a closed loop controller coupled to the processor, the controller configured to receive the position of the moveable partition and to control fluid delivery from the infusion pump based at least in part upon the position of the moveable partition.

33. The system of claim 24, wherein the infusion pump is an electrokinetic infusion pump.
